(19) United States
(12) Patent Application Publication
(10) Pub. No.: US 2026/0191403 A1
(43) Pub. Date: Jul. 9, 2026

(54) SYSTEMS AND METHODS FOR UTILIZING DATA FROM TRANSLUMINAL PROCEDURES

(71) Applicant: Edwards Lifesciences Corporation, Irvine, CA (US)

(72) Inventor: Bezalel Haberman-Browns, Pardes Hanna (IL)

(21) Appl. No.: 19/551,434

(22) Filed: Feb. 26, 2026

Related U.S. Application Data
(63) Continuation of application No. PCT/US2024/043953, filed on Aug. 27, 2024.
(60) Provisional application No. 63/579,157, filed on Aug. 28, 2023.

(51) Int. Cl. A61B 1/005; A61B 1/00

(52) U.S. Cl. CPC A61B 1/0057; A61B 1/00097

(57) ABSTRACT

A tool has a distal part that is adapted to be transluminally advanced toward a tissue of a subject. The tool comprises an extracorporeal unit, coupled to a proximal part of the tool, the extracorporeal unit comprising a mechanical control element (MCE), operably coupled to the distal part of the tool such that movement of the MCE mechanically manipulates the distal part of the tool. The tool also comprises an output device and a sensor, the sensor configured to: (i) sense the movement of the MCE, and (ii) output, via the output device, data indicative of the sensed movement of the MCE.

### CROSS-REFERENCES TO RELATED APPLICATIONS

The present application is a Continuation of International Patent Application PCT/US2024/043953 to Haberman-Browns, filed August 27, 2024, and titled "Systems and methods for utilizing data from transluminal procedures," which published as WO 2025/049422, and which claims the benefit of U.S. Patent Application No. 63/579,157, filed on August 28, 2023, and titled "Systems and methods for utilizing data from transluminal procedures."

Each of the above disclosures is incorporated by reference for all purposes in its entirety.

### BACKGROUND

Transcatheter procedures are technically challenging to perform; thus, the learning curve tends to be slow, and the training process lengthy. Additionally, large variations in human anatomy and pathology may render the procedure challenging and difficult, prolonging the time under anesthesia and resulting in tiring of the treating personnel. For these and other reasons, patients may not receive the desired results. Such challenges may be particularly great with transluminal, transcatheter procedures to repair or replace a cardiac valve.

### SUMMARY OF THE INVENTION

This summary is meant to provide some examples and is not intended to limit the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure may be included in the examples summarized here.

Some of the systems, apparatuses, devices, methods, techniques, etc. described herein, and implementations and applications thereof, include or are configured to be used with an encoder(s), e.g., a rotary or optical encoder, to record and/or control the movement of rotary parts of endoscopic catheter handles. Rotary encoders are electromechanical feedback devices used in a variety of industrial applications to provide information on position, speed, count or direction of motors. They can track motor shaft rotation to generate digital position and motion information, and are also used to control speed in multiple axes of motion.

In some implementations, encoders can be used as sensors on mechanical control elements (MCEs) of surgical tools, e.g., transluminal catheters, to provide information regarding a procedure performed using the tool. In some implementations, the encoder and/or the sensor can output data regarding movement of an MCE to which it is operationally coupled.

In some implementations, the output data can be collected by an output device, for concurrent or post-operative analysis.

In some implementations, a data-processing system (DPS) can then be used to temporally couple the sensor data with steps of the procedure, enabling a virtual reconstruction of the procedure.

In accordance with some implementations, a system and/or an apparatus, usable and/or for use with a real or simulated subject, the system/apparatus including a tool that includes an elongated shaft, and/or extracorporeal unit. The elongated shaft can extend proximally from a distal part of the tool.

In some implementations, the elongated shaft can be configured to be transluminally advanced toward a real or simulated tissue of the subject.

In some implementations, the extracorporeal unit can be coupled to the shaft at a proximal part of the tool. In some implementations, the extracorporeal unit can include a mechanical control element (MCE), an output device, and/or a sensor.

In some implementations, the MCE can be operably coupled to the distal part of the tool via the shaft, such that movement of the MCE mechanically manipulates the distal part of the tool.

In some implementations, the sensor can be configured to sense the movement of the MCE, and/or to output, via the output device, data indicative of the sensed movement of the MCE.

In some implementations, the distal part of the tool is sterilized.

In some implementations, the MCE is sterilized.

In some implementations, the sensor is sterilized.

In some implementations, the tissue is a real or simulated tissue of a heart of the subject, and/or the elongated shaft is configured to be transluminally advanced toward the tissue of the heart.

In some implementations, the tool is a cardiovascular repair tool.

In some implementations, the tool is a delivery tool for a cardiovascular implant.

In some implementations, the MCE includes a control knob.

In some implementations, the MCE includes a control lever.

In some implementations, the MCE includes a joystick.

In some implementations, the MCE is entirely mechanical.

In some implementations, the MCE is non-electronic.

In some implementations, the MCE comprises a wheel.

In some implementations, the MCE is configured to be operated manually.

In some implementations, the system/apparatus includes a device configured to be inserted through the tool.

In some implementations, the tool includes a hollow shaft, through which a device is configured to be inserted.

In some implementations, the elongated shaft includes a rod.

In some implementations, the elongated shaft includes a tube of a catheter, and/or the extracorporeal unit includes a handle of the catheter.

In some implementations, the MCE is operably coupled to the distal part of the tool such that movement of the MCE transluminally advances the distal part of the tool.

In some implementations, the MCE is operably coupled to the distal part of the tool such that movement of the MCE rotates the distal part of the tool.

In some implementations, the MCE is operably coupled to the distal part of the tool such that movement of the MCE deploys an implant from the tool.

In some implementations, the MCE is operably coupled to the distal part of the tool via one or more pull-wires, such that manipulation of the one or more pull-wires by the MCE is configured to steer the distal part of the tool.

In some implementations, the sensor includes a Hall effect sensor.

In some implementations, the sensor includes a potentiometer.

In some implementations, the output device is an electronic port.

In some implementations, the output device is a wireless transmitter.

In some implementations, the output device is a data recorder.

In some implementations, the tool includes a socket configured to receive a memory card, the output device being configured to output the data by writing the data to the memory card.

In some implementations, the MCE is mechanically coupled to the sensor.

In some implementations, the MCE is a hand-controlled MCE.

In some implementations, the MCE is operably coupled to the distal part of the tool such that movement of the MCE mechanically advances the distal part of the tool.

In some implementations, the MCE is operably coupled to the distal part of the tool such that movement of the MCE mechanically rotates the distal part of the tool.

In some implementations, the MCE is operably coupled to the distal part of the tool such that movement of the MCE mechanically bends the distal part of the tool.

In some implementations, the sensor is configured such that the output data is indicative of advancement of the distal part of the tool.

In some implementations, the sensor is configured such that the output data is indicative of bending of the distal part of the tool.

In some implementations, the sensor is configured such that the output data is indicative of a rotation of the distal part of the tool.

In some implementations, the sensor is configured to detect a direction of movement of the MCE.

In some implementations, the sensor is configured to detect an extent of movement of the MCE.

In some implementations, the sensor is configured to detect a rate of movement of the MCE.

In some implementations, the system/apparatus further includes a series of implants, wherein the tool further includes a second sensor, configured to sense passage of each of the implants past the second sensor, and/or to output, via the output device, data indicative of the sensed passage.

In some implementations, the apparatus further includes a series of implants, each of the implants housed by a respective cartridge that is mounted on the extracorporeal unit, and/or the tool further includes at least one cartridge sensor, configured to, for each of the cartridges, detect a position of the respective cartridge.

In some implementations, the system/apparatus further includes a series of implants, each of the implants housed by a respective cartridge that is mounted on the extracorporeal unit, and/or the tool further includes at least one cartridge sensor, configured to, for each of the respective cartridges, detect release of the respective anchor from the cartridge.

In some implementations, the extracorporeal unit includes a start button, operation of which outputs, via the output device, a start signal.

In some implementations, the extracorporeal unit includes a stop button, operation of which stops output of data via the output device.

In some implementations, the sensor includes an encoder.

In some implementations, the encoder is a rotary encoder.

In some implementations, the encoder is a linear encoder.

In some implementations, the encoder is an optical encoder.

In some implementations, the encoder is an incremental encoder.

In some implementations, the encoder is an absolute encoder.

In some implementations, the system/apparatus further includes a series of implants, each of the implants housed by a respective cartridge in a housing that is mounted on the extracorporeal unit, and/or the tool further includes at least one cartridge sensor, configured to, for each of the cartridges, detect a state of the cartridge.

In some implementations, the state of the cartridge defines a presence or an absence of an anchor within the cartridge.

In some implementations, for each of the cartridges, the at least one cartridge sensor includes an electrical contact.

In some implementations, for each of the cartridges, the at least one cartridge sensor includes a magnet.

In some implementations, for each of the cartridges, the at least one cartridge sensor includes a marker.

In some implementations, the system/apparatus further includes a tether anchored distally to a first implant, and/or stored proximally on a spring-powered winch.

In some implementations, the system/apparatus further includes a winch sensor configured to sense a length of tether released from the winch.

In some implementations, the spring-powered winch is configured to maintain tension on the tether.

In some implementations, the system/apparatus further includes a winch sensor configured to sense the tension on the tether.

In some implementations, the winch sensor includes a piezoelectric element.

In some implementations, the sensor is a first sensor, and the tool further includes a driver, advanceable through the elongated shaft.

In some implementations, a second sensor, configured to sense passage of the driver past the second sensor, and to output, via the output device, data indicative of the sensed passage.

In some implementations, the system/apparatus further includes a series of implants, the driver being configured to advance each implant of the series through the elongated shaft.

In some implementations, the second sensor is a component of the extracorporeal unit, and/or is configured to detect passage of the driver into the elongated shaft.

In some implementations, the driver is an anchor driver.

In some implementations, the second sensor further including a counter, configured to count instances of passage of the driver past the second sensor.

In some implementations, the counter is configured to output, via the output device, data indicative of the sensed passage.

In some implementations, the MCE is a first MCE, the sensor is a first sensor, and/or the extracorporeal unit further includes: a second MCE, operatively coupled to the distal part of the tool, such that movement of the second MCE mechanically manipulates the distal part of the tool in a manner different to that of the first MCE.

In some implementations, a second sensor, configured to sense the movement of the second MCE, and to output, via the output device, data indicative of the sensed movement of the second MCE.

In some implementations, the sensed movement of the first MCE includes bending of the distal part of the tool in a first direction.

In some implementations, the sensed movement of the second MCE includes bending of the distal part of the tool in a second direction.

In some implementations, the sensed movement of the first MCE includes rotation of the distal part of the tool.

In some implementations, the sensed movement of the second MCE includes axial advancement of the distal part of the tool.

In some implementations, the extracorporeal unit further includes: (i) a third MCE, operatively coupled to the distal part of the tool, such that movement of the third MCE mechanically manipulates the distal part of the tool in a manner different to that of both the first MCE and the second MCE; and/or (ii) a third sensor, configured to sense the movement of the third MCE, and to output, via the output device, data indicative of the sensed movement of the third MCE.

In some implementations, the sensed movement of the first MCE includes advancement of the distal part of the tool.

In some implementations, the sensed movement of the second MCE includes bending of the distal part of the tool in a first direction.

In some implementations, the sensed movement of the third MCE includes bending of the distal part of the tool in a second direction.

In some implementations, the system/apparatus is usable and/or for use with a data-processing system (DPS), and the output device is configured to output the data to the DPS.

In some implementations, the MCE is not configured to be controlled by the DPS.

In some implementations, the tool is not configured to be controlled by the DPS.

In some implementations, the system/apparatus further includes a data-processing system (DPS), configured to receive the data from the output device.

In some implementations, the DPS is not configured to provide feedback to the tool.

In some implementations, the DPS is not configured to control the tool.

In some implementations, the tool is configured to be usable and/or for use by an operator, and the DPS is not configured to provide feedback to the operator.

In some implementations, the system/apparatus further includes a timer.

In some implementations, the timer outputs, via the output device, a time-stream.

In some implementations, the timer is configured to, for each sensed movement of the MCE, associate, with the output of the data indicative of the sensed movement of the MCE, a timestamp indicative of timing of the sensed movement.

In some implementations, the timer is operatively coupled to the sensor.

In some implementations, the timer is operatively coupled to the output device.

In some implementations, the timer is configured to commence timing responsively to a first movement of the MCE.

In accordance with some implementations, a method including, while an operator, during a real or simulated medical procedure on a real or simulated subject, manipulates a distal part of a tool within a real or simulated subject by mechanically moving a first mechanical control element (MCE) and a second MCE disposed on an extracorporeal handle of the tool, sensing, via a first sensor, the movement of the first MCE, and/or sensing, via a second sensor, the movement of the second MCE.

The method can further include outputting first data indicative of the sensed movement of the first MCE, and/or second data indicative of the sensed movement of the second MCE.

The method can further include, within a data-processing system: receiving the first output data and the second output data; receiving time data; and/or using the time data, organizing a dataset in which timing of the first output data is temporally aligned with timing of the second output data.

In some implementations, the method further comprises sterilizing the tool prior to the medical procedure.

In some implementations, the medical procedure is a transcatheter procedure including delivery of implants to a heart of a subject, and using the time data comprises using the time data to reconstruct a sequence of delivering the implants to the heart.

In some implementations, the first MCE is movable to control steering of the distal part of the tool, and the second MCE is movable to control deployment of an implant from the tool, such that organizing the dataset comprises organizing the data set to represent timing of the steering with respect to timing of the deployment.

In some implementations, sensing the movement of the first MCE provides an indication of advancement of the distal part of the tool.

In some implementations, sensing the movement of the second MCE provides an indication of rotation of the distal part of the tool.

In some implementations, sensing the movement of the first MCE provides an indication of bending of the distal part of the tool.

In some implementations, recording the timing of the sensed movement is performed by a timer operationally coupled to the MCE.

In some implementations, recording the timing of the sensed movement is performed by a timer operationally coupled to the sensor.

In some implementations, organizing the data set is performed such that the operator does not receive feedback from the data-processing system while performing the procedure.

In some implementations, the MCE is controlled manually by the operator, such that sensing the movement of the MCE includes sensing the manual movement of the MCE by the operator.

In some implementations, the MCE is a first MCE, the sensor is a first sensor, and/or the extracorporeal handle further includes: a second MCE, operatively coupled to the distal part of the tool, such that movement of the second MCE mechanically manipulates the distal part of the tool in a manner different to that of the first MCE.

In some implementations, a second sensor, configured to sense the movement of the second MCE, and to output, via the output device, data indicative of the sensed movement of the second MCE.

In some implementations, outputting as output data the sensed movement and the timing of the sensed movement includes outputting as output data the sensed movement and the timing of the sensed movement of the first MCE, and the sensed movement and the timing of the sensed movement of the second MCE.

In some implementations, the method further includes a step of analyzing, by the data-processing system, the output data.

In some implementations, analyzing the output data is performed such that the operator does not receive feedback from the data-processing system while performing the procedure.

In some implementations, analyzing the output data includes providing information for improving future procedures performed using the method.

In some implementations, providing information includes providing at least one information type selected from the group consisting of: (i) timing of advancement of the distal part of the tool; (ii) sequence of advancement, bending, and rotation of the distal part of the tool; (iii) timing of delivery of a series of implants to a real or simulated tissue of the subject; and/or (iv) a number of implants delivered to the tissue.

In some implementations, providing information includes providing information to a manufacturer of the tool.

In some implementations, providing information includes providing information to the operator after completion of the procedure.

In accordance with some implementations, a system for performing a real or simulated procedure on a real or simulated tissue of a real or simulated subject includes a tool that includes an elongated shaft, an extracorporeal unit, and/or a data-processing system (DPS). In some implementations, the elongated shaft can extend proximally from a distal part of the tool, and can be configured to be transluminally advanced toward the tissue.

In some implementations, the extracorporeal unit can be coupled to the shaft at a proximal part of the tool, and/or can be operably coupled, via the shaft, to the distal part of the tool. In some implementations, the extracorporeal unit can include at least one mechanical control element (MCE), movement of which mechanically manipulates a component of the tool.

In some implementations, the extracorporeal unit can include a sensor, configured to sense the movement of the MCE during the procedure, and/or to output data indicative of the sensed movement of the MCE.

The DPS can be configured to receive the data output from the sensor, and/or generate a virtual reconstruction of the procedure based on timing of the received data.

In some implementations, the distal part of the tool is sterilized.

In some implementations, the MCE is sterilized.

In some implementations, the sensor is sterilized.

In some implementations, the system further includes at least a second MCE.

In some implementations, the system further includes at least a third MCE.

In some implementations, the sensed movement of the MCE provides an indication of linear advancement of the distal part of the tool.

In some implementations, the sensed movement of the MCE provides an indication of a rate of linear advancement of the distal part of the tool.

In some implementations, the sensed movement of the MCE provides an indication of rotation of the distal part of the tool.

In some implementations, the sensed movement of the MCE provides an indication of bending of the distal part of the tool.

In some implementations, the sensed movement of the MCE provides an indication of deployment of an implant along the elongated shaft.

In some implementations, an extent of the sensed movement is indicative of a position of the distal part of the tool.

In some implementations, the sensor is part of the tool.

In some implementations, the component of the tool is the distal part of the tool.

In some implementations, the component of the tool is the proximal part of the tool.

In some implementations, the component of the tool is the extracorporeal unit.

In some implementations, the component of the tool is a cradle for holding the proximal part of the tool.

In some implementations, the component of the tool is a holder for advancing the proximal part of the tool along a rail.

In some implementations, the component of the tool is a start button.

In some implementations, the component of the tool is a stop button.

In some implementations, the system further includes an anchor driver.

In some implementations, the anchor driver includes an anchor-deployment trigger and a sensor, the sensor configured to: (i) sense triggering of the anchor-deployment trigger during the procedure, and/or (ii) output data indicative of the sensed triggering.

In some implementations, the anchor-deployment trigger is configured to deploy an anchor into the tissue.

In some implementations, the component of the tool is a cartridge-release button.

In some implementations, the cartridge-release button is configured to release an anchor from its cartridge on the proximal part of the tool.

In some implementations, the DPS is configured to, responsively to receiving the output data indicative of the sensed movement of the MCE, output an indication of a timing of the sensed movement of the MCE.

In some implementations, the timing of the sensed movement is indicative of accomplishment of a specific part of the procedure.

In some implementations, the DPS is further configured to perform a comparison between the virtual reconstruction and a procedure model.

In some implementations, the DPS is further configured to update the procedure model responsively to the comparison.

In some implementations, the DPS is further configured to generate guidance responsively to the comparison.

In some implementations, the DPS is further configured to, responsively to the comparison, indicate discrepancies between the virtual reconstruction and the procedure model.

In accordance with some implementations, a computer-implemented method includes receiving first output data from a first sensor configured to sense mechanical movement of a first mechanical control element (MCE) on a surgical tool system used for performing a real or simulated transcatheter procedure; receiving second output data from a second sensor configured to sense mechanical movement of a second MCE on the surgical tool system; and/or receiving time data.

The method can further include, using the time data, generating a virtual reconstruction of the procedure including a dataset in which timing of the first output data is temporally aligned with timing of the second output data.

In accordance with some implementations, a data-processing system includes a processor configured to perform the steps of the method of paragraph 159.

In accordance with some implementations, a computer program includes instructions which, when the program is executed by a computer, cause the computer to carry out the steps of the method of paragraph 159. In some implementations, the computer program is stored in a non-transitory, computer-readable medium.

In some implementations, the procedure is a simulated procedure and generating the virtual reconstruction comprises generating the virtual reconstruction of the simulated procedure.

In some implementations, the transcatheter procedure includes delivery of implants to a heart of a subject, and using the time data comprises using the time data to reconstruct a sequence of delivering the implants to the heart.

In some implementations, the method further includes a step of suggesting adjustments to the surgical tool system.

In some implementations, the method further includes a step of subsequently suggesting improvements in technique to an operator who performed the procedure.

In some implementations, the method further includes a step of subsequently suggesting improvements in an algorithm for generating the virtual reconstruction.

In some implementations, the method further includes a step of highlighting incidents within the data.

In some implementations, the method further includes a step of highlighting patterns of interest within the data.

In some implementations, the method further includes a step of aggregating data from a multiplicity of virtual reconstructions.

In some implementations, the method further includes a step of generating a procedure model based on the aggregated data.

In some implementations, the method further includes a step of suggesting improvements to a procedure model based on the aggregated data.

In some implementations, the method further includes a step of suggesting improvements in technique to an operator based on the aggregated data.

In some implementations, generating the procedure model comprises cross-referencing the aggregated data to preprocedural planning.

In some implementations, generating the procedure model comprises cross-referencing the aggregated data to pre-operative medical imaging.

In some implementations, generating the procedure model comprises cross-referencing the aggregated data to intra-procedural medical imaging.

In some implementations, the method further comprises a step of inputting the procedure model to a surgical robotic controller.

In some implementations, the method further comprises a step of configuring the surgical robotic controller to use the procedure model to deploy an implant.

In some implementations, the method further includes a step of performing a comparison between the virtual reconstruction and a procedure model.

In some implementations, the method further includes a step of suggesting improvements in technique to an operator based on the procedure model.

In some implementations, the procedure model includes a set of instructions for performing the procedure, such that performing a comparison includes comparing steps performed in the virtual reconstruction to the set of instructions for performing the procedure.

In some implementations, the method further includes a step of suggesting adjustments to the procedure model.

In some implementations, the method further includes a step of suggesting adjustments to the virtual reconstruction.

In some implementations, the method further includes a step of subsequently suggesting improvements to the set of instructions.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

The present invention will be more fully understood from the following detailed description of implementations thereof, taken together with the drawings, in which:

### BRIEF DESCRIPTION OF THE DRAWINGS

FIG. 1 shows an example system or apparatus for recording a transluminal procedure at a real or simulated tissue of a real or simulated subject;

FIGS. 2-3 show example implementations of tools, catheters, or other devices for deployment of implants used in performing transluminal cardiovascular repair procedures with relevant sensors for each MCE;

FIG. 4 shows an example trace of output data for a portion of such a representative procedure; and

FIG. 5 shows a schematic diagram for flow of information in an example implementation of the described methods.

### DETAILED DESCRIPTION OF EMBODIMENTS

In the following description, specific configurations and details are set forth in order to provide a thorough understanding of the different aspects of the disclosure. Implementations and applications of the devices, systems, and techniques described herein can include any variant in which an element is substituted with a functionally equivalent element.

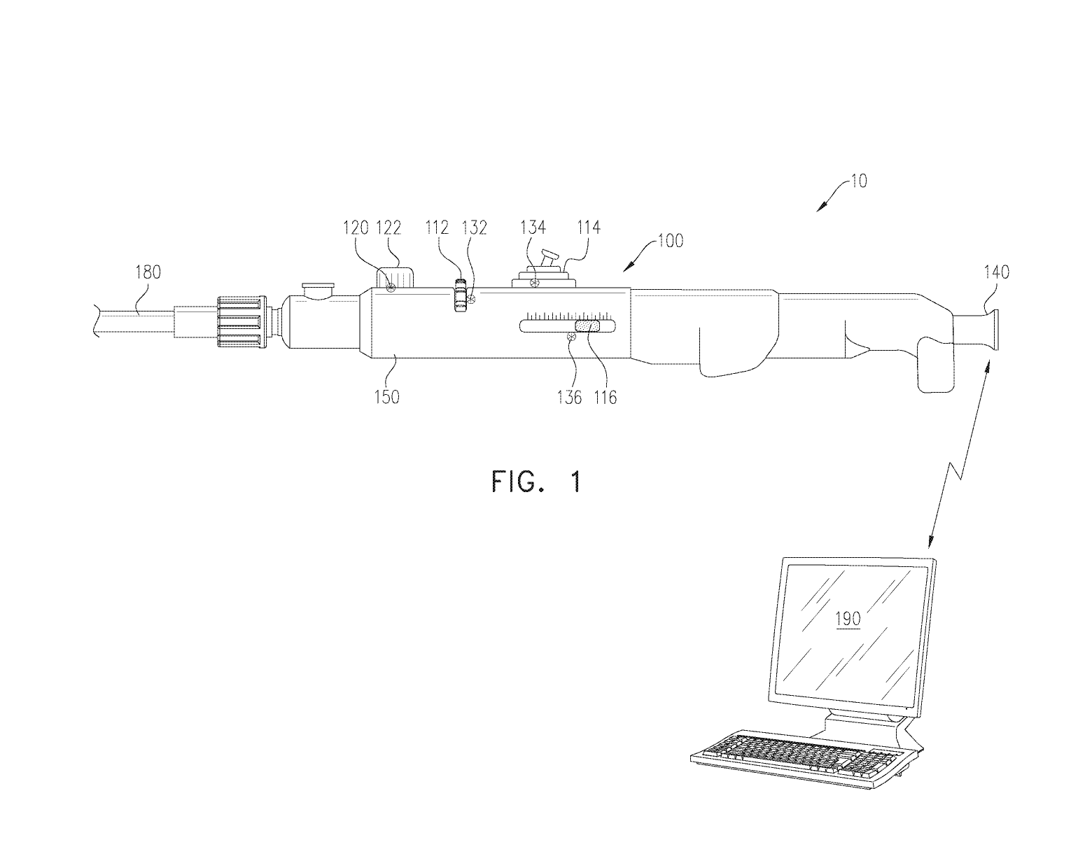

Reference is made to FIG. 1, which is a schematic illustration of a system 10 for performing a transluminal procedure on a subject, and for obtaining and/or recording data regarding the procedure, in accordance with some implementations. System 10 comprises a tool 100, e.g., a transluminal catheter, delivery system, endoscope, or similar. Tool 100 comprises one or more sensors (e.g., sensors 132, 134, 136) configured to detect use of the tool during the procedure, e.g., for obtaining and/or outputting data regarding the procedure. Such sensors can include, for example, rotary encoders, linear encoders, optical encoders, absolute encoders, incremental encoders, Hall effect sensors, potentiometers, or any other suitable sensors.

In some implementations, tool 100 may be identical to an existing tool (e.g., an existing catheter, delivery system, endoscope, or similar) except for the presence of sensors and their associated components, such as an output device (described hereinbelow). Moreover, tool 100 may advantageously be used by the operator (e.g., the physician) identically to an existing tool, e.g., without the sensors participating in the use of the tool, or in the procedure. During any given procedure, the operator performing that procedure typically receives no indication of the data obtained by the sensors. As described in more detail hereinbelow, the data obtained from the sensors can be used to learn how operators use the tool and/or perform the procedure, e.g., in order to modify training of operators, to modify the tool, and/or to modify the procedure performed using the tool.

Tool 100 has a proximal part comprising an extracorporeal unit 150. Tool 100 can have a shaft 180 (e.g., a tube or rod) extending distally from the extracorporeal unit to a distal part of the tool. Extracorporeal unit 150 can be operatively coupled to the distal part of the tool, e.g., via shaft 180. Shaft 180 is configured to be transluminally advanced via a lumen, e.g., a blood vessel, toward a tissue of a subject (e.g., a living subject, a simulation, etc.). Shaft 180 and/or the distal part of tool 100 can be configured to be advanced, rotated, or bent in one or more directions to enable navigation of the shaft through the lumen.

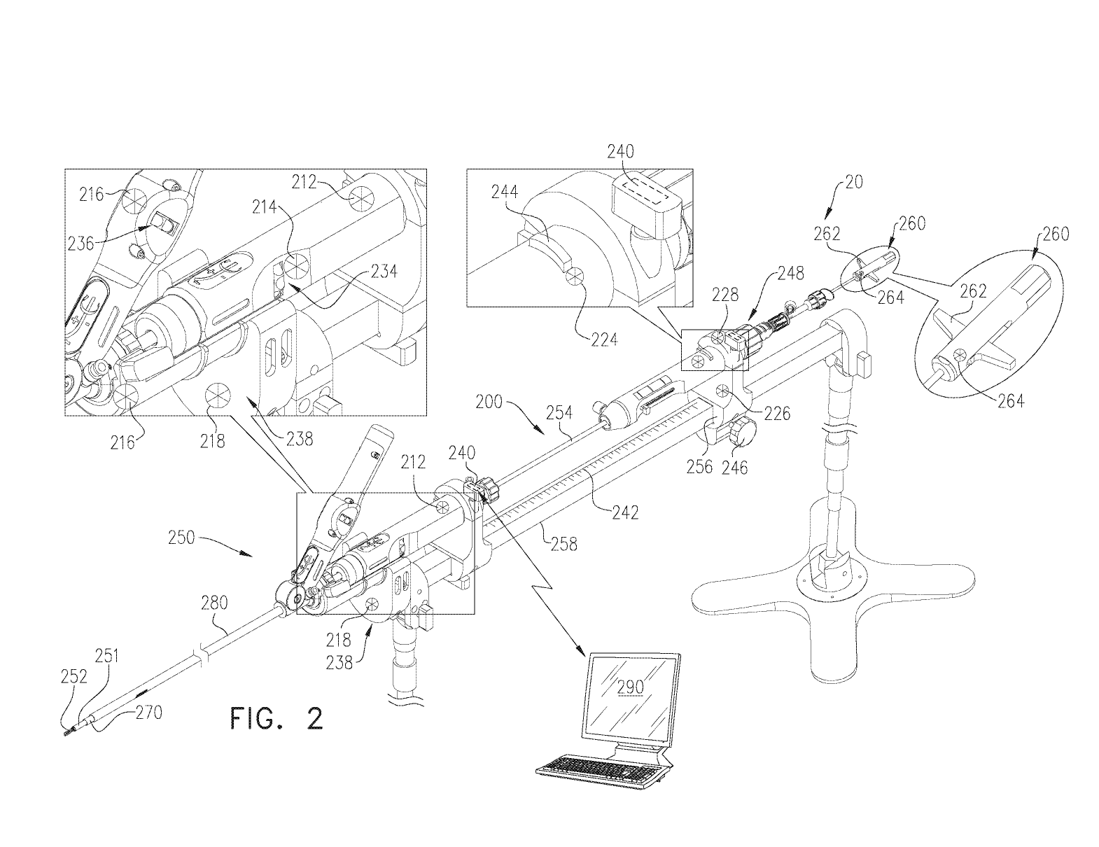

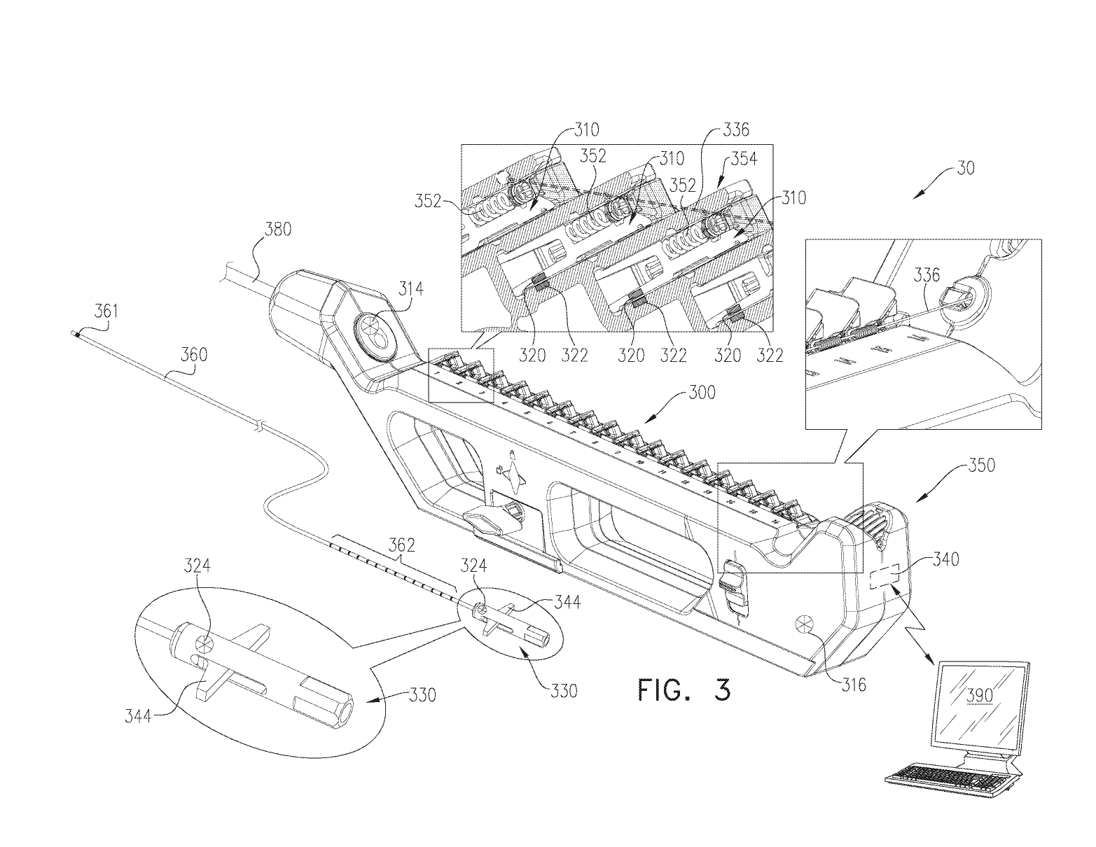

The description of components illustrated in FIG. 1 provides the location and function of these sensors in a somewhat generic manner (e.g., tool 100 is intended to be representative of a variety of tools), whereas FIGS. 2 and 3 provide specific examples of the use of such sensors in example systems and methods.

The distal part of tool 100 is manipulable using one or more mechanical control elements (MCEs) on extracorporeal unit 150 (or handle) of the tool/catheter. Each MCE is operably coupled to the distal part of the tool (e.g., via the shaft) such that movement of the MCE mechanically manipulates the distal part of the tool. Examples of such MCEs include rotatable knobs, sliding knobs, levers, trackballs, and joysticks. In some implementations, the MCEs may be operated manually, e.g., by grasping, pushing, or sliding by hand. In some implementations, the MCEs can be entirely mechanical, e.g., having no electrical, computerized, or robotic assistance.

One of the MCEs shown in the example of FIG. 1 is MCE 112, which comprises a control knob or wheel. In some implementations, MCE 112 can be operated by manual rotation. MCE 112 can, for example, be configured to facilitate an operator steering the distal part of tool 100, e.g., movement of the MCE by the operator can advance, rotate, or bend the distal part of shaft 180, such as by the MCE being connected to one or more pull-wires that extend along the shaft.

Tool 100 (e.g., extracorporeal unit 150) comprises a sensor 132 that is configured to sense the movement (e.g., direction of movement, extent of movement, rate of movement, and/or rotation) of MCE 112 (e.g., the movement that the operator causes in order to manipulate the distal part of tool 100). Sensor 132 can comprise an encoder (e.g., a rotary encoder). In some implementations, the sensor may comprise a mechanical, optical, magnetic, or electromagnetic induction type of sensor. The sensor may be configured to detect any type of movement, e.g., amount of rotation of the MCE, rotational speed of the MCE, rotational direction of the MCE, and/or rotational position of the MCE. Tool 100 (e.g., extracorporeal unit 150) can further comprise an output device 140, via which sensor 132 can be configured to output data indicative of the sensed movement of MCE 112.

Output device 140 can comprise an electronic port, a wireless transmitter, a data recorder, and/or a socket configured to receive a storage medium such as a memory card. In some implementations, e.g., implementations in which the output device comprises a socket, the output device can be configured to output the data by writing the data to the memory card.

In some implementations, extracorporeal unit 150 can comprise additional MCEs, e.g., a second MCE 114, and/or a third MCE 116, each configured to manipulate the distal part of tool 100 in a manner that differs from that performed by MCE 112.

MCE 114 can comprise a joystick or control lever. MCE 114 can be configured to facilitate an operator steering the distal part of tool 100.

In some implementations, movement (e.g., deflection) of MCE 114 can be detected by sensor 134. Sensor 134 may, for example, comprise a Hall-effect sensor, which uses magnets and electrical conductors to detect movement by measuring a change in voltage that occurs when a magnetic field interferes with electrical flows.

In some implementations, MCE 116 can comprise a sliding knob. Sensor 136 can be configured to detect movement (e.g., axial sliding) of the knob. For example, sensor 136 may comprise a linear encoder. MCE 116 may, for example, be configured to control advancement and retraction of a distal component of tool 100, or of an implant with respect to the tool.

For each MCE, the respective sensor can detect the direction, extent, and/or rate of movement of the MCE.

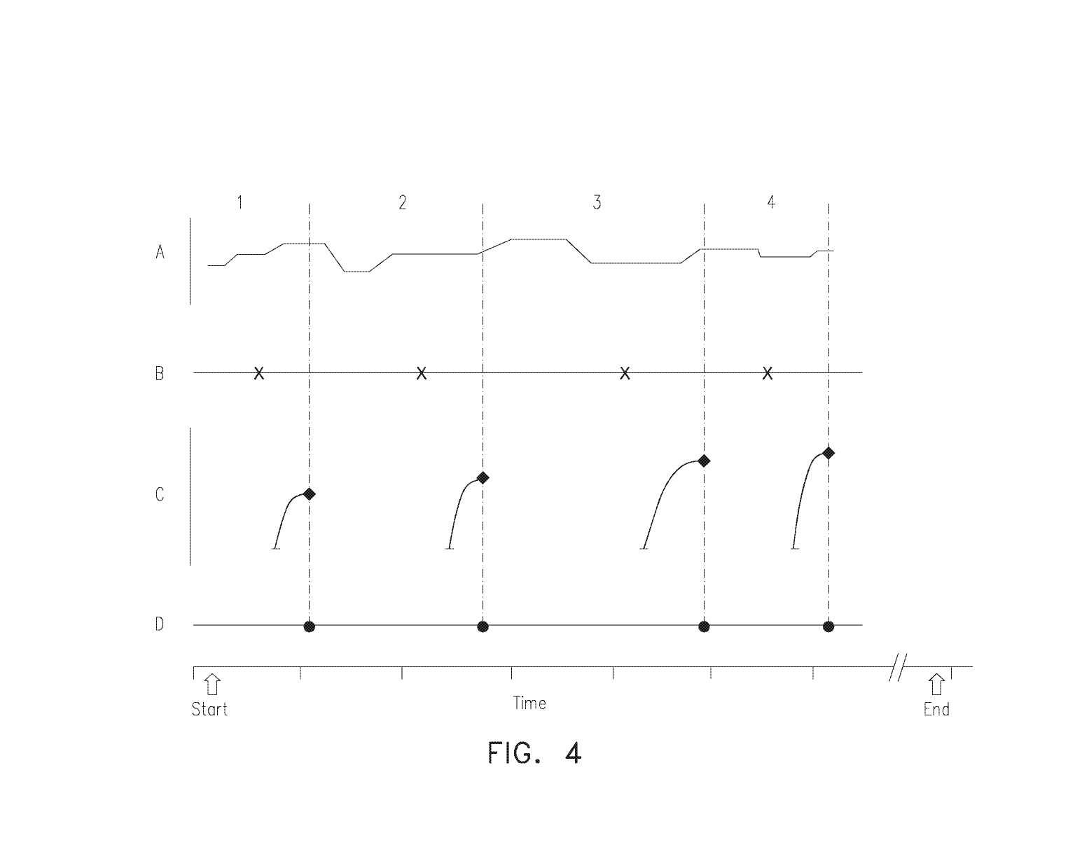

Output data may be indicative of various types of manipulation of the distal part of tool 100 achieved by MCE movement. For example, the output data can be indicative of bending, rotation, or advancement of the distal part of the tool. Output data can be transferred to a data-processing system (DPS) 190, configured to analyze the data, as further discussed herein below with respect to FIG. 4. This transfer can be an electronic (e.g., wired) or electromagnetic (e.g., wireless) transmission, or can be transfer of a storage medium such as a memory card. Although shown in FIG. 1, DPS 190 can be situated at a location distant from the site of the procedure being performed. For example, the operator performing the procedure with tool 100 may not need to be aware of DPS 190.

It is to be noted that extracorporeal unit 150 (and tool 100 more generally) is not configured to be controlled by DPS 190. Likewise, DPS 190 is not configured to provide feedback to the tool. Furthermore, DPS 190 may be configured to not provide information (e.g., feedback) to the human operator manipulating the tool – or at least not during the procedure. Rather, and as noted elsewhere herein, the data can be used only separately from (e.g., subsequently to) the procedure.

In some implementations, extracorporeal unit 150 comprises a start button 122, activation of which outputs a start signal via output device 140. For example, the operator may press the start button at the beginning of the procedure, the start signal therefore being indicative of the beginning of the procedure. For some implementations, start button 122 serves only this purpose (e.g., does not serve a purpose in the procedure itself). For some implementations, start button 122 unlocks (e.g., mechanically unlocks) the extracorporeal unit (e.g., serving as an interlock), e.g., such that tool 100 is maintained in a ready state until use. It is to be noted that, rather than a start button, a ripcord or similar can be used.

DPS 190 can be configured to begin obtaining (e.g., monitoring and/or recording) the output from output device 140 only once the start signal is received. DPS 190 can also begin timing the procedure at that point.

For some implementations, rather than a discrete start button 122 (or ripcord, or similar), extracorporeal unit 150 is configured to output the start signal upon movement (e.g., a first movement) of one of the MCEs (e.g., any of the MCEs, or a particular one of the MCEs).

In some implementations, the apparatus comprises a timer 120. The timer can be configured to output a time-stream with which the timing of events (e.g., outputs) during the procedure can be referenced. In some implementations, the time-stream comprises a simple record of incremental time passage throughout the procedure, e.g., output in parallel with the outputs of the sensors. In some implementations, the time-stream is integrated with the outputs of sensors 132 and/or sensor 134, such that each output of a sensor is associated with a timestamp indicative of timing of each sensed movement. For some implementations, timer 120 begins timing upon operation of start button 122. For some implementations, timer 120 begins timing upon movement (e.g., a first movement) of one of the MCEs (e.g., any of the MCEs, or a particular one of the MCEs).

Reference is now made to FIG. 2, which is a schematic illustration of a system 20 for performing a transluminal procedure on a subject, and for obtaining and/or recording data regarding the procedure, in accordance with some implementations. System 20 comprises a tool 200, comprising a proximal extracorporeal unit 250 and a shaft 254, extending distally from extracorporeal unit 250. Tool 200 can comprise a series of concentric catheters through which shaft 254 can be deployed. Shaft 254 can provide access for an anchor driver 260, configured to carry an implant 251 and anchor(s) 252 to a location in the cardiovascular tissue. Shaft 254 may control advancement of an implant with respect to (e.g., out of) tool 200 (e.g., catheter 270 thereof).

MCE 236 is configured to control lateral steering (e.g., deflection) of a distal part of an outermost catheter shaft 280, and can comprise a rotatable knob. MCE 234 is configured to control lateral steering (e.g., deflection) of a distal part of a middle catheter shaft 270, and can comprise a rotatable knob. MCE 248 controls deployment of implant 251 from tool 200. A cradle 238 is a system component within which tool 200 is mountable. For some implementations, cradle 238 enables rotation of tool 200 (e.g., extracorporeal unit 250) about its longitudinal axis within the cradle. MCE 246 can comprise a rotatable knob that facilitates advancement of extracorporeal unit 250 (or a part thereof) along a linear rail 258.

System 20, e.g., tool 200 thereof, can comprise a series of sensors configured to output data indicative of the sensed movement of various MCEs. For example, a sensor 214 can be configured to sense movement (e.g., rotation) of MCE 234. A sensor 216 can be configured to sense movement (e.g., rotation) of MCE 236. A sensor 228 can be configured to sense movement (e.g., rotation and/or axial translation) of MCE 248. Each of sensors 214, 216, and 228 can be configured to operate similarly to sensor 132 in FIG. 1, and is configured to provide a respective output via an output device 240 of extracorporeal unit 250. Output data can be transferred to a data-processing system (DPS) 290, configured to analyze the data, as further discussed herein with respect to FIGS. 1 and 4.

A sensor 212 can be configured to sense passage of anchors 252 past the sensor (e.g., through catheter 270), and to output, via output device 240, data indicative of the sensed passage. Anchors 252 can complete an electrical contact and/or comprise a magnetic component that is sensed by sensor 212.

Rail 258 can comprise or define a scale 242, paired with a sensor 226 mounted on a holder 256. Scale 242 and sensor 226 can therefore collectively serve as a linear encoder that detects a position of holder 256 along rail 258, e.g., responsively to operation of MCE 246.

Component 244 serves as an end-of-procedure button and sensor 224 is configured to sense depression of this button and to responsively output a stop signal. Similarly to start button 122, mutatis mutandis, in some implementations, component 244 is an MCE that serves a particular function in the procedure, such as serving as an interlock that must be released before final release of implant 251 from tool 200. Alternatively, component 244 can merely be an end-of-procedure button that serves only to provide a stop signal.

DPS 190 can be configured to stop obtaining (e.g., monitoring and/or recording) the output from output device 240 only once the stop signal is received.

Reference is additionally made to FIG. 4, which is a schematic illustration of representative output data that can be output from sensors described herein, according to some implementations. For example, the data shown can be representative of that which can be output by various sensors during a transluminal procedure via an output device. The graph can represent various types of data outputted by an output device, e.g., output device 140, 240, or 340. While the data are illustrative and can relate to various systems, for the purposes of the present discussion, the data represent output from sensors related to system 20, e.g., tool 200, in FIG. 2.

The traces presented in the graph represent data outputted via output device 240. For example, the data shown can represent the data as received by the DPS, or as arranged by the DPS. FIG. 4 shows data outputs A, B, C, and D synchronized with each other and juxtaposed with incremental units of time on a common x-axis. Time can be measured in seconds, minutes, or other meaningful increments that are relevant to the pace of the documented procedure.

The “start” indicator in FIG. 4 can represent receipt of a start signal, e.g., as described hereinabove.

Output A can be representative of the output of a sensor that provides a continuous output indicative of a status of its corresponding MCE. This can be appropriate, for example, for an MCE that continually affects the distal part of the tool, and/or has a plurality or continuum of states that correspond to a plurality or continuum of states of the distal part of the tool. Such an MCE can be, for example, a rotatable or slidable knob that can reside in a plurality or continuum of rotational or axial positions. Thus, the output line is slanted while the operator is moving the MCE (e.g., the knob), and becomes horizontal when the operator leaves the MCE in its current position. Moreover, the slant (or curve) of the line can be indicative of the rate at which the MCE was moved.

Manipulation of the MCE, e.g., the MCE whose movement is being displayed, can cause any necessary movement required to perform the procedure, such as rotation, bending, and/or advancing the distal part of the tool. Thus, with respect to the y-axis, an upward line segment indicates rotation or movement of the MCE in a first direction, and downward bending of the line represents rotation or movement of the MCE in a second, e.g., opposite, direction relative to the first direction. For example, output A can be from sensor 216, 214, or 228 (e.g., indicative of rotation of MCE 236, MCE 234, or MCE 248, respectively) of tool 200, or of sensor 132 or 136 (e.g., indicative of rotation of MCE 112 or sliding of MCE 116, respectively) of tool 100. The pattern of upward, downward, and horizontal line segments can represent the induced state (e.g., curvature) of the shaft of the tool as the operator navigates it through vasculature of the subject. The full data for an actual procedure can include multiple outputs similar to output A, from the respective sensors of several MCEs used for guiding the tool to its intended deployment location.

Outputs B and D can be representative of the sensing of discrete events that occur during the procedure. For example, output B can be representative of the output of sensor 212 as it senses passage of each anchor 252 past the sensor, as it is moved distally for advancement through the tool toward the target tissue. Similarly, output D can be representative of the output of sensor 264 as MCE 262 is used to release each anchor from driver 260. Thus, in FIG. 4, each event in output B is followed by an event in output D, e.g., the advancement of an anchor past sensor 212 is followed by the release of that anchor from driver 260. Outputs B and D will be discussed in greater detail herein below with respect to FIG. 3.

Reference is now made to FIG. 3, which is a schematic illustration of a system 30 for performing a transluminal procedure on a subject, and for obtaining and/or recording data regarding the procedure, in accordance with some implementations. System 30 comprises a tool 300, comprising a proximal extracorporeal unit 350 and a shaft 380, extending distally from extracorporeal unit 350. System 30, e.g., tool 300 thereof, is compatible with and can be used as part of system 20 of FIG. 2. Specifically, shaft 380, attached to extracorporeal unit 350, can be advanced, in place of shaft 254, through catheters 270 and 280. Tool 300, e.g., extracorporeal unit 350 thereof, can hold a series of implants or anchors 352, each of which is housed within a respective cartridge 310 of the tool. Cartridges 310 are fitted within a housing 354 of extracorporeal unit 350. System 30, e.g., tool 300, can comprise a driver 330, having a driveshaft 360 advanceable through shaft 380. Anchor driver 330 is configured to pick up anchor 352 from its respective cartridge 310, and transport it into and through the lumen of shaft 380, and through the vasculature of the subject, to an implant site on cardiovascular tissue.

Anchors 352 can be coupled to (e.g., threaded onto) a tether 336 that will serve as part of an eventual implant. As the first anchor 352 is advanced through shaft 380, it carries with it the distal end of the tether, and anchoring of the first anchor to tissue anchors the distal end of the tether to the tissue. As each subsequent anchor is advanced through shaft 380, it is slid along the tether toward the first anchor at the tissue. A proximal end of the tether can be stored on a spring-powered winch (not shown) within extracorporeal unit 350, which lets out the tether as necessary. During the letting out of the tether, and during the advancement and anchoring of subsequent anchors, the spring-powered winch maintains a little tension on the tether in order to eliminate slack on the tether.

System 30, e.g., tool 300 thereof, can comprise a series of sensors configured to output data indicative of the sensed movement of various MCEs. Tool 300 (e.g., extracorporeal unit 350) can further comprise an output device 340, via which sensors can be configured to output data indicative of the sensed movement of MCEs to which they are linked. Output data can be transferred to a data-processing system (DPS) 390, configured to analyze the data. The output data can be displayed, e.g., as shown in FIG. 4.

Tool 300, e.g., extracorporeal unit 350, can comprise a sensor 314 configured to detect discrete events of passage of driver 330 (e.g., a head 361 of the driver) and/or anchor 352 past the sensor. Sensor 314 can be configured to output, via an output device 340, data indicative of the sensed passage. This data can be represented, e.g., by output B in FIG. 4.

In some implementations, sensor 314 can be configured to sense advancement of driveshaft 360, e.g., a distance that the driveshaft has been advanced through shaft 380. For example, driveshaft 360 can have a series of contacts 362, magnets, or markers, disposed on the driveshaft, which can be sensed as a scale of advancement. For some such implementations, output C can represent the output of sensor 314, indicative of continuous distal-ward movement of driveshaft 360 through shaft 380, and thereby through the vasculature of the subject toward a target tissue. In output C, the y-axis represents distance advanced, such that the shape of each curve represents distance over time. A steeper rise of a curve corresponds to more rapid movement, whereas a nearly flat area of a curve indicates, e.g., that the operator has advanced the device nearly to the desired anchoring location and is slowly finalizing a site for deploying an anchor 352. The distance at which a given anchor is anchored is represented by the diamond at the end of each curve. For some implementations, this is simply derived from another output, such as the output from sensor 324, which senses actuation of MCE (e.g., trigger) 344, which is indicative of release of the anchor 352 from driver 330.

In the example shown, contacts 362 are disposed only along a proximal part of driveshaft 360, and therefore sensing of advancement only occurs for the distal-most part of the advancement (e.g., once the contacts reach sensor 314). In output C, the short horizontal line indicates the advancement point at which sensor 314 begins to sense advancement. The empty area temporally preceding each curve traced in output C represents proximal advancement and/or retraction of the intraluminal device, movement which may not be specifically tracked.

Tool 300 can further comprise one or more cartridge sensors 322. Cartridge sensors 322 can be configured to detect a position of cartridge 310 in a resting state, e.g., prior to release of each implant 352 from cartridge 310. In some implementations, cartridge sensor 322 can detect a state of the cartridge, e.g., containing an anchor or not containing an anchor. In some implementations, cartridge sensor 322 can be configured to detect release of each respective anchor 352 from its cartridge.

To release a given anchor 352 from its cartridge 310, the operator engages head 361 of driveshaft 360 with the anchor, pulls the anchor proximally. The cartridge 310 housing that anchor responsively slides out of housing 354, exposing the anchor and allowing it to be removed from the cartridge and advanced into shaft 380. In the example shown, each cartridge sensor 322 is disposed on housing 354, and senses contact with one or more electrical contacts 320 on cartridge 310. The sliding of cartridge 310 out of housing 354 disconnects the pair of electrical contacts 320, which is sensed by sensor 322. The output of sensor 322 can comprise discrete events. For example, output B can be representative of this output.

Similarly, output D can be representative of the output of sensor 324 (or sensor 264) as MCE 344 (or MCE 262), e.g., a trigger is actuated in order to release each anchor from driver 330 (or driver 260). The circles in output D correspond temporally to the diamonds in output C, illustrating that the distal-most advancement of the intraluminal device corresponds to the deployment or implantation site of the implant or anchor into the tissue.

Tool 300 can further comprise a sensor 316 (e.g., at the spring-powered winch), configured to sense tension on tether 336 and/or the length of the tether that has been let out from the winch. Sensor 316 can comprise a piezoelectric element. Output A can be representative of the type of data outputted by sensor 316.

In some implementations, sensor 316, or another sensor disposed near the spring-powered winch, can be configured to sense the length of tether 336 released during the procedure.

Reference is again made to FIGS. 1-3. In some implementations, and as mentioned with respect to FIG. 1, the DPS, e.g., DPS 190, 290, and/or 390 is located off-site, e.g., at a location distant from the site of the procedure being performed. For example, the operator performing the procedure may not need to be aware of the DPS. As such, the sensor and other data of the procedure outputted by the output device (e.g., recorded on a memory card), can be uploaded and/or received by the DPS only after completion of the procedure.

Referring again to FIG. 4, the starting point indicated can be defined by a start signal, e.g., as described hereinabove. For example, the start signal can be provided automatically by a first movement of an MCE, or can be provided by manual activation of a start button. Additionally or alternately, the start signal can be indirectly activated by an element of a timer, as further described elsewhere herein. The end of the procedure indicated can be defined by a stop signal, e.g., as described hereinabove. For example, the stop signal can be provided by activation of an end-of-procedure button, such as component 244.

In general, and as described hereinabove, FIG. 4 represents different types of output that can be provided by different types of sensors. Some types of data indicate discrete events, e.g., anchor release or shaft entrance to a catheter, whereas other data provide a continuous record of activity, e.g., turning a knob or advancing a shaft. The following paragraph is an alternative description of FIG. 4, as representing data from a given procedure performed using system 30, in which output A represents the output of a sensor of a steering knob; output B represents the output of sensors 322 detecting withdrawal of each anchor 352 from its cartridge 310; output C represents the output of sensor 314 detecting advancement of driveshaft 360; and output D represents the output of sensor 324 detecting actuation of trigger 344 releasing each anchor.

FIG. 4 contains a series of vertical panels (numbered 1–4), delineated by dotted vertical lines. Each panel includes data pertaining to deployment and anchoring of an individual anchor 352. The dotted vertical lines delineating the panels are coincident with the release of each anchor. Thus, the four panels of the graph represent information pertaining to the sequential implantation of four anchors. In the example shown, panel 2 is narrower than panel 3, indicating that the amount of time taken to fully deploy the second anchor was shorter than the time taken to fully deploy the third anchor.

Thus, the relative time between the diamond in panel 1 representing final deployment of a first anchor, and the initial appearance of the curve in output C, representing the distal ward extension of a second anchor, together with other data, provides useful information regarding the timing of each anchor deployment event.

The data provided by the sensors described in FIGS. 1-4 can be used in several ways to improve outcomes of transluminal, transcatheter procedures on the cardiovascular system.

Such procedures require long training periods and a high degree of skill to acquire proficiency. New users of a given transcatheter system performing a specific procedure can be trained directly on human subjects by experienced operators. Alternatively or additionally, in some cases, users are trained by practicing on a virtual model of the procedure to be performed, under human and/or computer-assisted supervision.

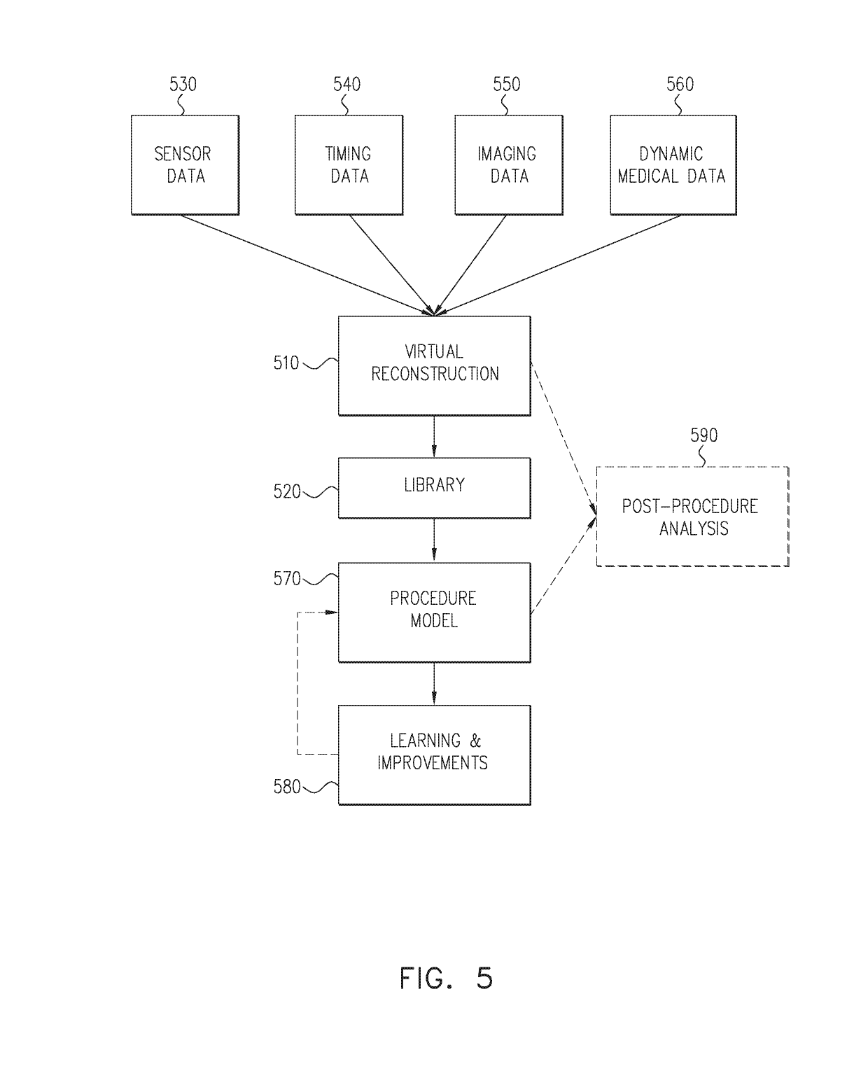

Reference is now made to FIG. 5, which is a schematic that shows, inter alia, types of data that can be collected during a procedure, and possible ways in which this data can be used to improve training and future performances of the procedure, e.g., by inputting the sensor and other data to a virtual procedure model.

The sensor data can be used as an integral part of refining the procedure model. These data can provide advice during virtual training to new users on how to navigate challenging parts of the procedure. Such advice may speed up the learning curve for the procedure and/or can be fed into training tools to provide information when asked by the user-in-training for suggestions on how to proceed. Such advice in training may expand the population of health-care professionals capable of conducting such complex procedures by providing the tools and assistance to better train less-skilled users. Secondly, data acquired in previous procedures could be shared, either individually or in aggregate, with the operators, and displayed while the operators perform subsequent procedures, to provide guidance for their movements. In some implementations, a trainee can practice a procedure virtually on a specific patient's anatomy. For reference, catheter manipulations of previous attempts by the same trainee can be semi-translucently overlayed on the current simulation. Thirdly, the data can be shared with research and development teams to improve and optimize subsequent generations of the device and its associated procedures, based on actual clinical usage and feedback from data sensors.

In some implementations, FIG. 5 represents one or more computer-implemented methods. In some implementations, steps of and/or instructions for the computer-implemented methods can be stored in a non-transitory, computer-readable medium. In some implementations, the steps and/or instructions can be executable using computer hardware.

In some implementations, the data can be used to generate a virtual reconstruction 510 of the procedure. As discussed in FIGS. 1-4 above, data can be collected from MCE sensors, depicted as sensor data 530. Timing data 540 is shown as being provided separately from sensor data 530 (e.g., provided by a discrete timer, or by the DPS). However, as noted hereinabove, an alternative to this option is that the timing data can be included in the output of one or more of the sensors (e.g., within sensor data 530). In some implementations, different types of data can also be used to generate virtual reconstruction 510, including imaging data 550, and dynamic medical data 560. Imaging data that can be collected include, but are not limited to, angiography, fluoroscopy, and trans-esophageal echocardiography (TEE). Dynamic medical data 560 include vital signs and other subject data collected during the procedure, including but not limited to heart rate, blood pressure, respiratory rate, ECG traces, oxygen saturation, and core temperature. These data can be recorded by the anesthesiologist during the course of the procedure and later inputted, or can be directly outputted from the respective data-acquisition device (e.g., imaging device, medical monitor device, etc.) to the DPS.

In some implementations, the DPS processes the various types of data pertaining to the procedure, e.g., sorts and/or temporally aligns the data (e.g., as shown in a limited manner for sensor data 530 in FIG. 4), to generate the virtual reconstruction 510. In some implementations, the virtual reconstruction provides co-incident timing of each data point, such that it is possible to juxtapose specific events. For example, if the distal end of a tool causes irritation to a blood vessel lining, thereby inducing a hypotensive or tachycardiac response at a particular point of the procedure, the virtual reconstruction would provide an indication of the synchronous timing of these events.

In some implementations, in further processing steps, the DPS described above, or some differing implementation of the DPS, can store the reconstructed data in a library 520 of virtual reconstructions of a particular type of procedure, e.g., a cardiac valve repair or replacement, using the same or similar system and equipment used to perform the procedure for which virtual reconstruction 510 was generated. Such libraries can be kept in cloud storage and accessed remotely by multiple users and/or multiple DPSs. The libraries can be created for various procedures performed by any number of systems, e.g., are not limited to a particular procedure or tool.

Virtual reconstruction 510 can be analyzed either or both individually and/or in aggregate, as part of multiple virtual procedures 510 performed by various operators on different patients. The analysis of virtual reconstructions 510 in aggregate, e.g., as part of library 520, can be used to generate a procedure model 570 (e.g., a hypothetical ideal or standardized model) with standardized optimal MCE movements intended to successfully carry out the procedure. In some implementations, long-term follow-up data on each subject can be incorporated into procedure model 570 to provide recommendations and advice for improving performance of the procedure, and/or system components.

Based on ongoing additions of virtual reconstructions 510 into library 520, procedure model 570 can be continually updated. These updates can be used to provide post-procedure analysis 590 for any given virtual reconstruction 510, e.g., providing specific advice to improve a given procedure. For analysis of individual procedures, algorithms can be used to identify maneuvers of the operator that were performed optimally and/or according to plan, and those that were performed suboptimally. For example, the DPS may rank each MCE movement according to a scale of performance. Such a scale may relate to the timing of the movement, success of anchor implantation, and/or other aspects of procedure completion.

Procedure model 570 can also be used as a basis for learning and improvements 580. Improvements can take a number of forms. For example, this can include adjustments to guidance for training new operators; adjustments to the procedure model, based on, e.g., excellent technique exhibited by a particular operator, or a repeated point of difficulty experienced by many operators; and/or adjustments to the tool and/or system used to perform the procedure. In some implementations, the DPS highlights incidents and/or patterns of interest within the data, responsively to which humans can determine appropriate adjustments to model 570.

Alternatively or additionally, the DPS can provide recommendations of such adjustments to model 570, or can simply make such adjustments. For example, model 570 can be generated and/or refined via machine learning. In some implementations, if a maneuver carried out during a procedure generates an output that agrees with model 570, the machine learning algorithm can reinforce the corresponding part of the model, whereas if the output from the maneuver differs from the model, this can be flagged for review and/or the machine learning algorithm can adjust the corresponding part of the model accordingly.

In some implementations, an initial procedure model is generated (e.g., taught) from an initial set of data. For example, a set of initial performances of the procedure may be performed by one or more experts, and the data outputted from those initial performances may be used to generate the initial procedure model, e.g., by training a machine learning algorithm on the data.

In some implementations, the computer-implemented method(s) shown in FIG. 5 can be initially run in a “shadow mode” in which post-procedure analysis 590 is not provided. Rather, while in “shadow mode,” learning & improvements 580 are performed without any feedback to the operator. Once it has been determined that procedure model 570 is sufficiently accurate and reliable, post-procedure analyses 590 can begin to be provided. That is, following sufficient training and testing in shadow mode, it may be determined that the procedure model is sufficiently updated that it can be made available to provide real-time feedback to human operators and/or to system developers.

In some implementations, the procedure model may be cross-referenced to preprocedural planning, to pre-operative medical imaging, e.g., CT, and/or to intra-procedural medical imaging, e.g., fluoroscopy and/or echocardiography. For example, the number of implants required for a specific procedure may be estimated from pre-operative imaging, and that information programmed into the procedure model. Deviations from the plan may be adjusted intra-operatively, based on fluoroscopy, and the procedure model again adjusted accordingly.

In some implementations, the procedure model can be used to direct a robot to take the place of a human operator in carrying out some steps of the procedure. In such a scenario, the procedure model based on actual navigational data may be fed into, e.g., a surgical robotic controller, that would be able to navigate and deploy the implant without external output. For example, movement of one or more MCEs may be automated, such that a robotic controller is used to maneuver the catheter. The robotic controller can be linked via a DPS to the procedure model. Thus, steps of the procedure indicated by the procedure model are carried out by robotically controlled hardware connected to the DPS.

The described systems, apparatuses, devices, methods, etc. should not be construed as limiting in any way. Instead, the present disclosure is directed toward all novel and nonobvious features and aspects of the various disclosed implementations and applications, alone and in various combinations and sub-combinations with one another. The disclosed systems, apparatuses, devices, methods, etc. are not limited to any specific aspect, feature, or combination thereof, nor do the disclosed systems, apparatuses, devices, methods, etc. require that any one or more specific advantages be present or problems be solved.

The systems (and/or components thereof) and techniques described herein can be used in combination with, and/or to facilitate, any of those described in any of the following references, each of which is incorporated herein by reference in its entirety:

International Patent Application No. PCT/IL2013/050860 to Sheps et al., filed 23 October 2013, and titled “Controlled steering functionality for implant-delivery tool”, which published as WO 2014/064694;

International Patent Application No. PCT/US2021/042056 to Conklin et al., filed July 16, 2021, and titled “Adjustable annuloplasty ring and delivery system”, which published as WO 2022/026219;

International Patent Application No. PCT/IB2021/058665 to Halabi et al., filed September 23, 2021, and titled “Anchor magazines”, which published as WO 2022/064401;

International Patent Application No. PCT/IB2022/051099 to Shafigh et al., filed February 8, 2022, and titled “Tissue anchors and techniques for use therewith”, which published as WO 2022/172149; and

International Patent Application No. PCT/IB2023/062298 to Halabi et al., filed December 6, 2023, and titled “Annuloplasty implants and systems for use therewith”, which published as WO 2024/121770.

For some implementations, and as shown, the DPS is, or is a component of, a discrete (e.g., purpose-made) device. For some implementations, the DPS is a general-purpose DPS (e.g., a processor of a general-purpose computer) programmed to receive and/or analyze the output data.

In the present disclosure, the term DPS can refer to, be part of, or include an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor (shared, dedicated, group) that executes code; memory (shared, dedicated, or group) that stores code executed by a processor; other suitable hardware components, such as optical, magnetic, or solid state drives, that provide the described functionality; or a combination of some or all of the above, such as in a system-on-chip. The term algorithm, as used above, can include code, software, firmware, and/or microcode, and can refer to programs, routines, codes, functions, classes, and/or objects. The term library encompasses a group memory that, in combination with additional memories, stores some or all algorithms relevant to one or more procedure models. The term memory can be subset of the term computer-readable medium. Non-limiting examples of a non-transitory tangible computer readable medium include nonvolatile memory, volatile memory, magnetic storage, and optical storage.

Any of the various systems, assemblies, devices, components, apparatuses, etc. in this disclosure can be sterilized (e.g., with heat, radiation, ethylene oxide, hydrogen peroxide, etc.) to ensure they are safely useable and/or for use with patients, and the methods herein can comprise (or additional methods comprise or consist of) sterilization of the associated system, device, component, apparatus, etc. (e.g., with heat, radiation, ethylene oxide, hydrogen peroxide, etc.). The scope of the present disclosure includes, in some implementations, sterilizing one or more of any of the various systems, devices, apparatuses, etc. in this disclosure.

Any of the techniques, methods, operations, steps, etc. described or suggested herein or in the references incorporated herein can be performed on a living subject (e.g., human, other animal, etc.) or on a simulation, such as a cadaver, cadaver heart, simulator, imaginary person, etc. When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

### Example Implementations (some non-limiting examples of the concepts herein are recited below):

Example 1. Apparatus, usable and/or for use with a real or simulated subject, the apparatus comprising a tool that comprises an elongated shaft extending proximally from a distal part of the tool, and configured to be transluminally advanced toward a tissue of the subject. The tool may further comprise an extracorporeal unit, coupled to the shaft at a proximal part of the tool. The extracorporeal unit may comprise a mechanical control element (MCE), operably coupled to the distal part of the tool via the shaft, such that movement of the MCE mechanically manipulates the distal part of the tool. The extracorporeal unit may comprise an output device; and/or a sensor. The sensor may be configured to sense the movement of the MCE, and/or output, via the output device, data indicative of the sensed movement of the MCE.

Example 2. The apparatus according to example 1, wherein the distal part of the tool is sterilized.

Example 3. The apparatus according to any one of examples 1–2, wherein the MCE is sterilized.

Example 4. The apparatus according to any one of examples 1–3, wherein the sensor is sterilized.

Example 5. The apparatus according to any one of examples 1–4, wherein the tissue is a tissue of a heart of the real or simulated subject, and wherein the elongated shaft is configured to be transluminally advanced toward the tissue of the heart.

Example 6. The apparatus according to any one of examples 1–5, wherein the tool is a cardiovascular repair tool.

Example 7. The apparatus according to any one of examples 1–6, wherein the tool is a delivery tool for a cardiovascular implant.

Example 8. The apparatus according to any one of examples 1–7, wherein the MCE comprises a control knob.

Example 9. The apparatus according to any one of examples 1–8, wherein the MCE comprises a control lever.

Example 10. The apparatus according to any one of examples 1–9, wherein the MCE comprises a joystick.

Example 11. The apparatus according to any one of examples 1–10, wherein the MCE is entirely mechanical.

Example 12. The apparatus according to any one of examples 1–11, wherein the MCE is non-electronic.

Example 13. The apparatus according to any one of examples 1–12, wherein the MCE comprises a wheel.

Example 14. The apparatus according to any one of examples 1–13, wherein the MCE is configured to be operated manually.

Example 15. The apparatus according to any one of examples 1–14, wherein the apparatus comprises a device configured to be inserted through the tool.

Example 16. The apparatus according to any one of examples 1–15, wherein the tool comprises a hollow shaft, through which a device is configured to be inserted.

Example 17. The apparatus according to any one of examples 1–16, wherein the elongated shaft comprises a rod.

Example 18. The apparatus according to any one of examples 1–17, wherein the elongated shaft comprises a tube of a catheter, and the extracorporeal unit comprises a handle of the catheter.

Example 19. The apparatus according to any one of examples 1–18, wherein the MCE is operably coupled to the distal part of the tool such that movement of the MCE transluminally advances the distal part of the tool.

Example 20. The apparatus according to any one of examples 1–19, wherein the MCE is operably coupled to the distal part of the tool such that movement of the MCE rotates the distal part of the tool.

Example 21. The apparatus according to any one of examples 1–20, wherein the MCE is operably coupled to the distal part of the tool such that movement of the MCE deploys an implant from the tool.

Example 22. The apparatus according to any one of examples 1–21, wherein the MCE is operably coupled to the distal part of the tool via one or more pull-wires, such that manipulation of the one or more pull-wires by the MCE is configured to steer the distal part of the tool.

Example 23. The apparatus according to any one of examples 1–22, wherein the sensor comprises a Hall effect sensor.

Example 24. The apparatus according to any one of examples 1–23, wherein the sensor comprises a potentiometer.

Example 25. The apparatus according to any one of examples 1–24, wherein the output device is an electronic port.

Example 26. The apparatus according to any one of examples 1–25, wherein the output device is a wireless transmitter.

Example 27. The apparatus according to any one of examples 1–26, wherein the output device is a data recorder.

Example 28. The apparatus according to any one of examples 1–27, wherein the tool comprises a socket configured to receive a memory card, the output device being configured to output the data by writing the data to the memory card.

Example 29. The apparatus according to any one of examples 1–28, wherein the MCE is mechanically coupled to the sensor.

Example 30. The apparatus according to any one of examples 1–29, wherein the MCE is a hand-controlled MCE.

Example 31. The apparatus according to any one of examples 1–30, wherein the MCE is operably coupled to the distal part of the tool such that movement of the MCE mechanically advances the distal part of the tool.

Example 32. The apparatus according to any one of examples 1–31, wherein the MCE is operably coupled to the distal part of the tool such that movement of the MCE mechanically rotates the distal part of the tool.

Example 33. The apparatus according to any one of examples 1–32, wherein the MCE is operably coupled to the distal part of the tool such that movement of the MCE mechanically bends the distal part of the tool.

Example 34. The apparatus according to any one of examples 1–33, wherein the sensor is configured such that the output data is indicative of advancement of the distal part of the tool.

Example 35. The apparatus according to any one of examples 1–34, wherein the sensor is configured such that the output data is indicative of bending of the distal part of the tool.

Example 36. The apparatus according to any one of examples 1–35, wherein the sensor is configured such that the output data is indicative of a rotation of the distal part of the tool.

Example 37. The apparatus according to any one of examples 1–36, wherein the sensor is configured to detect a direction of movement of the MCE.

Example 38. The apparatus according to any one of examples 1–37, wherein the sensor is configured to detect an extent of movement of the MCE.

Example 39. The apparatus according to any one of examples 1–38, wherein the sensor is configured to detect a rate of movement of the MCE.

Example 40. The apparatus according to any one of examples 1–39, further comprising a series of implants, wherein the tool further comprises a second sensor, configured to sense passage of each of the implants past the second sensor, and to output, via the output device, data indicative of the sensed passage.

Example 41. The apparatus according to any one of examples 1–40, further comprising a series of implants, each of the implants housed by a respective cartridge that is mounted on the extracorporeal unit, and wherein the tool further comprises at least one cartridge sensor, configured to, for each of the cartridges, detect a position of the respective cartridge.

Example 42. The apparatus according to any one of examples 1–41, further comprising a series of implants, each of the implants housed by a respective cartridge that is mounted on the extracorporeal unit, and wherein the tool further comprises at least one cartridge sensor, configured to, for each of the respective cartridges, detect release of the respective anchor from the cartridge.

Example 43. The apparatus according to any one of examples 1–42, wherein the extracorporeal unit comprises a start button, operation of which outputs, via the output device, a start signal.

Example 44. The apparatus according to any one of examples 1–43, wherein the extracorporeal unit comprises a stop button, operation of which stops output of data via the output device.

Example 45. The apparatus according to any one of examples 1–44, wherein the sensor comprises an encoder.

Example 46. The apparatus according to example 45, wherein the encoder is a rotary encoder.

Example 47. The apparatus according to example 45, wherein the encoder is a linear encoder.

Example 48. The apparatus according to example 45, wherein the encoder is an optical encoder.

Example 49. The apparatus according to example 45, wherein the encoder is an incremental encoder.

Example 50. The apparatus according to example 45, wherein the encoder is an absolute encoder.

Example 51. The apparatus according to any one of examples 1–50, further comprising a series of implants, each of the implants housed by a respective cartridge in a housing that is mounted on the extracorporeal unit, and wherein the tool further comprises at least one cartridge sensor, configured to, for each of the cartridges, detect a state of the cartridge.

Example 52. The apparatus according to example 51, wherein the state of the cartridge defines a presence or an absence of an anchor within the cartridge.

Example 53. The apparatus according to example 51, wherein, for each of the cartridges, the at least one cartridge sensor comprises an electrical contact.

Example 54. The apparatus according to example 51, wherein, for each of the cartridges, the at least one cartridge sensor comprises a magnet.

Example 55. The apparatus according to example 51, wherein, for each of the cartridges, the at least one cartridge sensor comprises a marker.

Example 56. The apparatus according to example 51, further comprising a tether anchored distally to a first implant, and stored proximally on a spring-powered winch.

Example 57. The apparatus according to example 56, further comprising a winch sensor configured to sense a length of tether released from the winch.

Example 58. The apparatus according to example 56, wherein the spring-powered winch is configured to maintain tension on the tether.

Example 59. The apparatus according to example 58, further comprising a winch sensor configured to sense the tension on the tether.

Example 60. The apparatus according to example 59, wherein the winch sensor comprises a piezoelectric element.

Example 61. The apparatus according to any one of examples 1–60, wherein the sensor is a first sensor, and the tool further comprises: a driver, advanceable through the elongated shaft, and/or a second sensor, configured to sense passage of the driver past the second sensor, and to output, via the output device, data indicative of the sensed passage.

Example 62. The apparatus according to example 61, further comprising a series of implants, the driver being configured to advance each implant of the series through the elongated shaft.

Example 63. The apparatus according to example 61, wherein the elongated shaft comprises a tube of a catheter, the extracorporeal unit comprises a handle of the catheter, and the second sensor is a component of the extracorporeal unit and/or an extracorporeal portion of the catheter, and is configured to detect passage of the driver into the elongated shaft.

Example 64. The apparatus according to example 61, wherein the driver is an anchor driver.

Example 65. The apparatus according to example 61, wherein the second sensor further comprises a counter, configured to count instances of passage of the driver past the second sensor.

Example 66. The apparatus according to example 65, wherein the counter is configured to output, via the output device, data indicative of the sensed passage.

Example 67. The apparatus according to any one of examples 1–66, wherein the MCE is a first MCE, and/or the sensor is a first sensor. The extracorporeal unit may further comprise a second MCE, operatively coupled to the distal part of the tool, such that movement of the second MCE mechanically manipulates the distal part of the tool in a manner different to that of the first MCE. The extracorporeal unit may further comprise a second sensor, configured to sense the movement of the second MCE, and to output, via the output device, data indicative of the sensed movement of the second MCE.

Example 68. The apparatus according to example 67, wherein the sensed movement of the first MCE comprises bending of the distal part of the tool in a first direction.

Example 69. The apparatus according to example 67, wherein the sensed movement of the second MCE comprises bending of the distal part of the tool in a second direction.

Example 70. The apparatus according to example 67, wherein the sensed movement of the first MCE comprises rotation of the distal part of the tool.

Example 71. The apparatus according to example 67, wherein the sensed movement of the second MCE comprises axial advancement of the distal part of the tool.

Example 72. The apparatus according to example 67, wherein the extracorporeal unit further comprises a third MCE, operatively coupled to the distal part of the tool, such that movement of the third MCE mechanically manipulates the distal part of the tool in a manner different to that of both the first MCE and the second MCE; and/or a third sensor, configured to sense the movement of the third MCE, and to output, via the output device, data indicative of the sensed movement of the third MCE.

Example 73. The apparatus according to example 72, wherein the sensed movement of the first MCE comprises advancement of the distal part of the tool.

Example 74. The apparatus according to example 72, wherein the sensed movement of the second MCE comprises bending of the distal part of the tool in a first direction.

Example 75. The apparatus according to example 72, wherein the sensed movement of the third MCE comprises bending of the distal part of the tool in a second direction.

Example 76. The apparatus according to any one of examples 1–75, wherein the apparatus is usable and/or for use with a data-processing system (DPS), and wherein the output device is configured to output the data to the DPS.

Example 77. The apparatus according to example 76, wherein the MCE is not configured to be controlled by the DPS.

Example 78. The apparatus according to any one of examples 76–77, wherein the tool is not configured to be controlled by the DPS.

Example 79. The apparatus according to any one of examples 1–78, further comprising a data-processing system (DPS), configured to receive the data from the output device.

Example 80. The apparatus according to example 79, wherein the DPS is not configured to provide feedback to the tool.

Example 81. The apparatus according to example 79, wherein the DPS is not configured to control the tool.

Example 82. The apparatus according to example 79, wherein the tool is configured to be usable and/or for use by an operator, and wherein the DPS is not configured to provide feedback to the operator.

Example 83. The apparatus according to any one of examples 1–82, further comprising a timer.

Example 84. The apparatus according to example 83, wherein the timer outputs, via the output device, a time-stream.

Example 85. The apparatus according to example 83, wherein the timer is configured to, for each sensed movement of the MCE, associate, with the output of the data indicative of the sensed movement of the MCE, a timestamp indicative of timing of the sensed movement.

Example 86. The apparatus according to example 83, wherein the timer is operatively coupled to the sensor.

Example 87. The apparatus according to example 83, wherein the timer is operatively coupled to the output device.

Example 88. The apparatus according to example 83, wherein the timer is configured to commence timing responsively to a first movement of the MCE.

Example 89. A method, comprising, while an operator, during a real or simulated medical procedure on a real or simulated subject, manipulates a distal part of a tool within the subject by mechanically moving a first mechanical control element (MCE) and a second MCE disposed on an extracorporeal handle of the tool. The method further includes sensing, via a first sensor, the movement of the first MCE, and/or sensing, via a second sensor, the movement of the second MCE. The method further comprises outputting (a) first data indicative of the sensed movement of the first MCE, and/or (b) second data indicative of the sensed movement of the second MCE. The method may further comprise, within a data-processing system, (a) receiving the first output data and the second output data; (b) receiving time data; and/or (c) using the time data, organizing a dataset in which timing of the first output data is temporally aligned with timing of the second output data.

Example 90. The method according to example 89, further comprising sterilizing the tool prior to the real or simulated medical procedure.

Example 91. The method according to any one of examples 89–90, wherein the real or simulated medical procedure is a transcatheter procedure including delivery of implants to a real or simulated heart of a real or simulated subject, and/or using the time data comprises using the time data to reconstruct a sequence of delivering the implants to the heart.

Example 92. The method according to example any one of examples 89–91, wherein the first MCE is movable to control steering of the distal part of the tool, and the second MCE is movable to control deployment of an implant from the tool, such that organizing the dataset comprises organizing the dataset to represent timing of the steering with respect to timing of the deployment.

Example 93. The method according to any one of examples 89–92, wherein sensing the movement of the first MCE provides an indication of advancement of the distal part of the tool.

Example 94. The method according to any one of examples 89–93, wherein sensing the movement of the second MCE provides an indication of rotation of the distal part of the tool.

Example 95. The method according to any one of examples 89–94, wherein sensing the movement of the first MCE provides an indication of bending of the distal part of the tool.

Example 96. The method according to any one of examples 89–95, wherein recording the timing of the sensed movement is performed by a timer operationally coupled to the MCE.

Example 97. The method according to any one of examples 89–96, wherein recording the timing of the sensed movement is performed by a timer operationally coupled to the sensor.

Example 98. The method according to any one of examples 89–97, wherein organizing the dataset is performed such that the operator does not receive feedback from the data-processing system while performing the real or simulated procedure.

Example 99. The method according to any one of examples 89–98, wherein the MCE is controlled manually by the operator, such that sensing the movement of the MCE comprises sensing the manual movement of the MCE by the operator.

Example 100. The method according to any one of examples 89–99, wherein the MCE includes a first MCE, and the sensor is a first sensor. The extracorporeal handle further comprises a second MCE, operatively coupled to the distal part of the tool, such that movement of the second MCE mechanically manipulates the distal part of the tool in a manner different to that of the first MCE. A second sensor is configured to sense the movement of the second MCE, and to output, via the output device, data indicative of the sensed movement of the second MCE. Outputting as output data the sensed movement and the timing of the sensed movement comprises outputting as output data the sensed movement and the timing of the sensed movement of the first MCE, and the sensed movement and the timing of the sensed movement of the second MCE.

Example 101. The method according to any one of examples 89–100, further comprising a step of analyzing, by the data-processing system, the output data.

Example 102. The method according to example 101, wherein analyzing the output data is performed such that the operator does not receive feedback from the data-processing system while performing the real or simulated procedure.

Example 103. The method according to example 101, wherein analyzing the output data comprises providing information for improving future real or simulated procedures performed using the method.

Example 104. The method according to example 103, wherein providing information comprises providing at least one information type selected from the group consisting of timing of advancement of the distal part of the tool; sequence of advancement, bending, and rotation of the distal part of the tool; timing of delivery of a series of implants to a tissue of the subject; and/or a number of implants delivered to the tissue.

Example 105. The method according to example 103, wherein providing information comprises providing information to a manufacturer of the tool.

Example 106. The method according to example 103, wherein providing information comprises providing information to the operator after completion of the real or simulated procedure.

Example 107. A system for performing a real or simulated procedure on a real or simulated tissue of a real or simulated subject comprising a tool, a sensor, and/or a data-processing system. The tool comprises an elongated shaft and an extracorporeal unit. The shaft extends proximally from a distal part of the tool, and is configured to be transluminally advanced toward the tissue. The extracorporeal unit is coupled to the shaft at a proximal part of the tool. The extracorporeal unit is operably coupled, via the shaft, to the distal part of the tool. The extracorporeal unit comprises at least one mechanical control element (MCE), movement of which mechanically manipulates a component of the tool. The sensor is configured to sense the movement of the MCE during the procedure, and/or output data indicative of the sensed movement of the MCE. The data-processing system (DPS) is configured to receive the data output from the sensor, and/or generate a virtual reconstruction of the real or simulated procedure based on timing of the received data.

Example 108. The apparatus according to example 107, wherein the distal part of the tool is sterilized.

Example 109. The apparatus according to any one of examples 107–108, wherein the MCE is sterilized.

Example 110. The apparatus according to any one of examples 107–109, wherein the sensor is sterilized.

Example 111. The system according to any one of examples 107–110, further comprising at least a second MCE.

Example 112. The system according to any one of examples 107–111, further comprising at least a third MCE.

Example 113. The system according to any one of examples 107–112, wherein the sensed movement of the MCE provides an indication of linear advancement of the distal part of the tool.

Example 114. The system according to any one of examples 107–113, wherein the sensed movement of the MCE provides an indication of a rate of linear advancement of the distal part of the tool.

Example 115. The system according to any one of examples 107–114, wherein the sensed movement of the MCE provides an indication of rotation of the distal part of the tool.

Example 116. The system according to any one of examples 107–115, wherein the sensed movement of the MCE provides an indication of bending of the distal part of the tool.

Example 117. The system according to any one of examples 107–116, wherein the sensed movement of the MCE provides an indication of deployment of an implant along the elongated shaft.

Example 118. The system according to any one of examples 107–117, wherein an extent of the sensed movement is indicative of a position of the distal part of the tool.

Example 119. The system according to any one of examples 107–118, wherein the sensor is part of the tool.

Example 120. The system according to any one of examples 107–119, wherein the component of the tool is the distal part of the tool.

Example 121. The system according to any one of examples 107–120, wherein the component of the tool is the proximal part of the tool.

Example 122. The system according to any one of examples 107–121, wherein the component of the tool is the extracorporeal unit.

Example 123. The system according to any one of examples 107–122, wherein the component of the tool is a cradle for holding the proximal part of the tool.

Example 124. The system according to any one of examples 107–123, wherein the component of the tool is a holder for advancing the proximal part of the tool along a rail.

Example 125. The system according to any one of examples 107–124, wherein the component of the tool is a start button.

Example 126. The system according to any one of examples 107–125, wherein the component of the tool is a stop button.

Example 127. The system according to any one of examples 107–126, further comprising an anchor driver.

Example 128. The system according to example 127, wherein the anchor driver comprises an anchor-deployment trigger and a sensor, the sensor configured to sense triggering of the anchor-deployment trigger during the real or simulated procedure, and/or output data indicative of the sensed triggering.

Example 129. The system according to example 128, wherein the anchor-deployment trigger is configured to deploy an anchor into the tissue.

Example 130. The system according to example 128, wherein the component of the tool is a cartridge-release button.

Example 131. The system according to example 130, wherein the cartridge-release button is configured to release an anchor from its cartridge on the proximal part of the tool.

Example 132. The system according to any one of examples 107–131, wherein the DPS is configured to, responsively to receiving the output data indicative of the sensed movement of the MCE, output an indication of a timing of the sensed movement of the MCE.

Example 133. The system according to example 132, wherein the timing of the sensed movement is indicative of accomplishment of a specific part of the real or simulated procedure.

Example 134. The system according to any one of examples 107–133, wherein the DPS is further configured to perform a comparison between the virtual reconstruction and a procedure model.

Example 135. The system according to example 134, wherein the DPS is further configured to update the procedure model responsively to the comparison.

Example 136. The system according to example 134, wherein the DPS is further configured to generate guidance responsively to the comparison.

Example 137. The system according to example 134, wherein the DPS is further configured to, responsively to the comparison, indicate discrepancies between the virtual reconstruction and the procedure model.

Example 138. A real or simulated computer-implemented method, comprising receiving first output data from a first sensor configured to sense mechanical movement of a first mechanical control element (MCE) on a surgical tool system used for performing a transcatheter procedure. The method further comprises receiving second output data from a second sensor configured to sense mechanical movement of a second MCE on the surgical tool system. The method further comprises receiving time data; and/or using the time data, generating a virtual reconstruction of the procedure comprising a dataset in which timing of the first output data is temporally aligned with timing of the second output data.

Example 139. A data-processing system comprising a processor configured to perform the steps of the method of example 138.

Example 140. A computer program comprising instructions which, when the program is executed by a computer, cause the computer to carry out the steps of the method of example 138.

Example 141. The method according to any one of examples 138–140, wherein the procedure is a simulated procedure and generating the virtual reconstruction comprises generating the virtual reconstruction of the simulated procedure.

Example 142. The method according to any one of examples 138–141, wherein the transcatheter procedure includes delivery of implants to a real or simulated heart of a real or simulated subject, and using the time data comprises using the time data to reconstruct a sequence of delivering the implants to the heart.

Example 143. The method according to any one of examples 138–142, further comprising a step of suggesting adjustments to the surgical tool system.

Example 144. The method according to any one of examples 138–143, further comprising a step of subsequently suggesting improvements in technique to an operator who performed the real or simulated procedure.

Example 145. The method according to any one of examples 138–144, further comprising a step of subsequently suggesting improvements in an algorithm for generating the virtual reconstruction.

Example 146. The method according to any one of examples 138–145, further comprising a step of highlighting incidents within the data.

Example 147. The method according to any one of examples 138–146, further comprising a step of highlighting patterns of interest within the data.

Example 148. The method according to any one of examples 138–147, further comprising a step of aggregating data from a multiplicity of virtual reconstructions.

Example 149. The method according to example 148, further comprising a step of suggesting improvements to a real or simulated procedure model based on the aggregated data.

Example 150. The method according to example 148, further comprising a step of suggesting improvements in technique to an operator based on the aggregated data.

Example 151. The method according to example 148, further comprising a step of generating a procedure model based on the aggregated data.

Example 152. The method according to example 151, wherein generating the procedure model comprises cross-referencing the aggregated data to preprocedural planning.

Example 153. The method according to example 151, wherein generating the procedure model comprises cross-referencing the aggregated data to pre-operative medical imaging.

Example 154. The method according to example 151, wherein generating the procedure model comprises cross-referencing the aggregated data to intra-procedural medical imaging.

Example 155. The method according to example 151, wherein the method further comprises a step of inputting the procedure model to a surgical robotic controller r.

Example 156. The method according to example 155, wherein the method further comprises a step of configuring the surgical robotic controller to use the procedure model to deploy an implant.

Example 157. The method according to any one of examples 138–150, further comprising a step of performing a comparison between the virtual reconstruction and a procedure model.

Example 158. The method according to example 157, further comprising a step of suggesting improvements in technique to an operator based on the procedure model.

Example 159. The method according to example 157, wherein the procedure model comprises a set of instructions for performing the procedure, such that performing a comparison comprises comparing steps performed in the virtual reconstruction to the set of instructions for performing the procedure.

Example 160. The method according to example 159, further comprising a step of suggesting adjustments to the procedure model.

Example 161. The method according to example 159, further comprising a step of suggesting adjustments to the virtual reconstruction.

Example 162. The method according to example 159, further comprising a step of subsequently suggesting improvements to the set of instructions. The present invention is not limited to the examples that have been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

Various implementations of systems, devices, methods, etc. are disclosed herein, and any combination of their features, components, and options can be made unless specifically excluded. For example, various descriptions of tools, can be used with any appropriate sensor, and/or delivered and implanted by any appropriate method, even if a specific combination is not explicitly described. Likewise, the different constructions and features of devices and systems can be mixed and matched, such as by combining any catheter type/feature, sensor type/feature, anatomical site, etc., even if not explicitly disclosed. In short, individual components of the disclosed systems can be combined unless mutually exclusive or physically impossible.

Although the operations of some of the disclosed methods are described in a particular, sequential order for convenient presentation, it should be understood that this manner of description encompasses rearrangement, unless a particular ordering is required by specific language set forth below. For example, operations described sequentially can in some cases be rearranged or performed concurrently. Moreover, for the sake of simplicity, the attached figures may not show the various ways in which the disclosed systems, apparatuses, devices, methods, etc. can be used in conjunction with other systems, apparatuses, devices, methods, etc.

1. An apparatus for use with a tissue of a subject, the apparatus comprising a tool that has a distal part that is adapted to be transluminally advanced toward the tissue, the tool comprising an extracorporeal unit, coupled to a proximal part of the tool, the extracorporeal unit comprising:
a mechanical control element (MCE), operably coupled to the distal part of the tool such that movement of the MCE mechanically manipulates the distal part of the tool;
an output device; and
a sensor, configured to:
sense the movement of the MCE, and
output, via the output device, data indicative of the sensed movement of the MCE.

2. The apparatus according to claim 1, wherein the apparatus further comprises an elongated shaft extending from the extracorporeal unit at the proximal part of the tool distally to the distal part of the tool, the shaft configured to facilitate the transluminal advancement of the distal part of the tool toward the tissue.

3. The apparatus according to claim 1, wherein the tissue is a tissue of a heart of the subject, and wherein the distal part is configured to be transluminally advanced toward the tissue of the heart.

4. The apparatus according to claim 1, wherein the MCE is non-electronic.

5. The apparatus according to claim 1, wherein the MCE is configured to be operated manually.

6. The apparatus according to claim 1, wherein the MCE is operably coupled to the distal part of the tool such that movement of the MCE transluminally advances the distal part of the tool.

7. The apparatus according to claim 1, wherein the MCE is operably coupled to the distal part of the tool such that movement of the MCE rotates the distal part of the tool.

8. The apparatus according to claim 1, wherein the MCE is operably coupled to the distal part of the tool such that movement of the MCE deploys an implant from the tool.

9. The apparatus according to claim 1, wherein the MCE is operably coupled to the distal part of the tool via one or more pull-wires, such that manipulation of the one or more pull-wires by the MCE is configured to steer the distal part of the tool.

10. The apparatus according to claim 1, wherein the MCE is mechanically coupled to the sensor.

11. The apparatus according to claim 1, wherein the MCE is a hand-controlled MCE.

12. The apparatus according to claim 1, wherein the MCE is operably coupled to the distal part of the tool such that movement of the MCE mechanically advances the distal part of the tool.

13. The apparatus according to claim 1, wherein the MCE is operably coupled to the distal part of the tool such that movement of the MCE mechanically rotates the distal part of the tool.

14. The apparatus according to claim 1, wherein the MCE is operably coupled to the distal part of the tool such that movement of the MCE mechanically bends the distal part of the tool.

15. The apparatus according to claim 1, wherein the sensor is configured such that the output data is indicative of advancement of the distal part of the tool.

16. The apparatus according to claim 1, wherein the sensor is configured such that the output data is indicative of bending of the distal part of the tool.

17. The apparatus according to claim 1, wherein the sensor is configured such that the output data is indicative of a rotation of the distal part of the tool.

18. The apparatus according to claim 1, wherein the sensor comprises a rotary encoder.

19. The apparatus according to claim 1, wherein:
the MCE is a first MCE,
the sensor is a first sensor, and
the extracorporeal unit further comprises:
a second MCE, operatively coupled to the distal part of the tool, such that movement of the second MCE mechanically manipulates the distal part of the tool in a manner different to that of the first MCE; and
a second sensor, configured to sense the movement of the second MCE, and to output, via the output device, data indicative of the sensed movement of the second MCE.

20. A system for performing a procedure on a tissue of a subject comprising:
a tool comprising:
an elongated shaft extending proximally from a distal part of the tool, and configured to be transluminally advanced toward the tissue; and
an extracorporeal unit:
coupled to the shaft at a proximal part of the tool;
operably coupled, via the shaft, to the distal part of the tool; and
comprising at least one mechanical control element (MCE), movement of which mechanically manipulates a component of the tool;
a sensor, configured to:
sense the movement of the MCE during the procedure, and
output data indicative of the sensed movement of the MCE;
a data-processing system (DPS) configured to:
receive the data output from the sensor, and
generate a virtual reconstruction of the procedure based on timing of the received data.
